(19) United States
(12) Patent Application Publication
(10) Pub. No.: US 2004/0170584 A1
(43) Pub. Date: Sep. 2, 2004

(54) Nail hardener

(76) Inventor: Christine Renard, Maintenon (FR)

Correspondence Address: YOUNG & THOMPSON, 745 SOUTH 23RD STREET 2ND FLOOR, ARLINGTON, VA, 22202

(21) Appl. No.: 10/483,059

(22) PCT Filed: Jul. 9, 2002
(86) PCT No.: PCT/FR02/02399

(30) Foreign Application Priority Data
Jul. 10, 2001 (FR) 01/09127

(51) Int. Cl. A61K 7/04

(52) U.S. Cl. 424/61

(57) ABSTRACT

The invention concerns the use of citral as nail hardener, The citral is preferably of natural origin, used in amounts less than 5 wt. % in a nail varnish, or in a varnish base layer or in a nail care base.

The present invention relates to a hardener compound for the nails, and its use in cosmetic compositions for the nails, particularly nail polish.

In the field of nail polish, a formol has until now been widely used in proportions of the order of 3 to 4% by weight to harden the nails.

However, this molecule is more and more frequently classified as products adapted to give rise to allergies, which is why it is now sought to limit its usage, even to replace it totally.

In research for active agents to harden the nails, equivalent to formol, the applicant has discovered that citral (3,7-dimethyl 2,6-octanedial) has the property of hardening the nails. Citral of natural origin in particular, a mixture of two isomers: geranial and neral, has this regenerative property for the structure of the nails.

This aldehyde reacts like formol to improve the structure of the nails, without having its toxicity.

Natural citral is extracted from essential oils of certain plants, among which are:

| | | | |
|---|---|---|---|
| Backhousia citriodora which | about | 95-97% | by weight of citral |
| contains | | | |
| Litsea citrata | ″ | 90% | ″ |
| Cymbopogon flexuosus | ″ | 75$ | ″ |
| Leaves of Leptospermum | ″ | 70-80% | ″ |
| liversidgei | | | |
| Litsea cubeba | ″ | 70% | ″ |
| Ocimum grotissimum | ″ | 66.5% | ″ |
| Lindera citriodora | ″ | 65% | ″ |
| Calypranthes parriculata | ″ | 62% | ″ |
| Leaves of Citrus aurantifolia | ″ | 36% | ″ |
| Green citron | ″ | 6-9% | ″ |
| Citron | ″ | 2-5% | ″ |

The above information is gathered from Fenaroli's Handbook of Flavor Ingredients, 1975, cited by Food Cosmet. Toxicol, vol 17, p. 259-266, 1979.

Until now, citral was used principally in the field of foods as a flavoring agent and in the field of cosmetics (soaps, creams, perfume bases) as a perfume, at concentrations varying approximately between 0.0002 and 0.8% by weight (see Table I).

TABLE I
Content of citral in various compositions (% by weight)
(Food Cosmet. Toxicol., vol 17, p. 259-266, 1979)

Concentration

| | | Washing | Creams, | |
|---|---|---|---|---|
| | Soaps | products | lotions | Perfumes |
| Conventional | 0.02 | 0.002 | 0.005 | 0.2 |
| Maximum | 0.2 | 0.02 | 0.02 | 0.8 |

The present invention thus relates to a new use of citral, namely as a hardener for the nails. Preferably, the citral is of natural origin, extracted from plants, the extract containing at least 2% by weight of citral, preferably at least 60%.

Preferably, the citral is thus used in a cosmetic composition for the nails, which can include up to 5% by weight of citral, preferably up to 0.01% by weight, or even only up to 0.05% by weight.

Preferably, the citral is used in a cosmetic composition for the nails not including any other aldehyde, and principally not including formol. This permits particularly avoiding all the toxicological or allergenic problems inherent in the use of formol.

In nail polish, different tests have shown that a low concentration, namely about 0.001 and 0.05%, can provide a substantial improvement in the physical aspect of the nails. The effect of citral shows up in the nails being less fragile, less breakable, having fewer cleavages, fewer fissures, greater thickness with a more regular surface. This improvement increases also with the concentration of citral.

The present invention relates particularly to the use of citral in a colored or uncolored nail polish, in a nail polish base layer, or else in a base for care of the nails.

The present invention also relates to a cosmetic composition for the nails including up to 0.05% by weight of citral.

The invention is illustrated by the following examples:

### EXAMPLES

### Example 1

Use of citral as a hardener in a colored nail polish.

In this nail polish, the citral used is an essential oil obtained from “Verveine of the Indies” (Cymbopogon citratus).

The nail polish has the following composition (by weight %):

| | |
|---|---|
| Butyl acetate | 32 |
| Ethyl acetate | 28.27 |
| Nitrocellulose | 12 |
| Polyester resin | 7 |
| Isopropyl alcohol | 5.2 |
| Acetyltributylcitrate | 5 |
| Tosylamide/epoxy resin | 4 |
| Stearalkonium hectorite | 1.1 |
| Styrene/acrylic resin | 1 |
| Benzophenone-1 | 0.1 |
| Dimethicone | 0.01 |
| Citral | 0.02 |
| Mica | 2 |
| DC Red 7 | 1.2 |
| DC Red 34 | 0.8 |
| Titanium dioxide | 0.3 |

This composition has been tested on a sampling of 21 persons for one month, under normal conditions of use (nail polish removed and reapplied every 2 to 4 days).

The results of the observations of the users are collected in Table II hereafter:

TABLE II
Number of subjects having noted an improvement
(number of subjects tested: 21)

| | | | | Very | |
|---|---|---|---|---|---|
| Improvement | Slight | Moderate | Substantial | substantial | Total |
| Nail | 4 | 3 | 3 | 4 | 14(67%) |
| fragility | | | | | |
| Broken | 7 | 2 | 1 | 5 | 15(71%) |
| nails | | | | | |
| Fissured | 5 | 1 | 4 | 4 | 14(67%) |
| nails | | | | | |
| Cleaved | 7 | 4 | 2 | 3 | 16(76%) |
| nails | | | | | |
| Thin nails | 8 | 2 | 2 | 4 | 16(76%) |
| Hardness of | 5 | 4 | 3 | 5 | 17(80%) |
| nails | | | | | |
| Nail | 3 | 1 | / | 2 | 6(29%) |
| surface | | | | | |

More than two-thirds of the subjects have noted an improvement in the appearance of their nails as to fragility and hardness of the nails, broken nails, fissured nails, cleaved nails, thin nails. Moreover, 29% of the subjects have noted an improvement of the contour of the surface of the nails.

These results prove that citral has properties of hardening the nails even at a content as low as 0.02% by weight in a nail polish.

### Example 2

In this uncolored nail polish, the citral used is obtained by distillation of the oil of Litsea Cubeba. This fraction contains 92% citral.

| | |
|---|---|
| Butyl acetate | 35 |
| Ethyl acetate | 33.85 |
| Cellulose acetobutyrate | 7 |
| Nitrocellulose | 5 |
| Polyester resin | 8 |
| Acetyltributylcitrate | 6 |
| Tosylamide/epoxy resin | 5 |
| Benzophenone-1 | 0.1 |
| Dimethicone | 0.01 |
| Citral | 0.04 |

Tests carried out under conditions identical to those of Example 1 have shown that the number of satisfied persons remains the same, with a greater proportion of very satisfied persons.

1. Use of citral as a hardener for the nails.

2. Use according to claim 1, characterized in that the citral is of natural origin.

3. Use according to claim 2, characterized in that the citral is extracted from plants, containing at least 2% by weight of citral.

4. Use according to one of claims 1 to 3 in a cosmetic composition for the nails.

5. Use according to claim 4, characterized in that the composition includes up to 5% by weight of citral.

6. Use according to claim 5, characterized in that the composition includes up to 0.01% by weight of citral.

7. Use according to claim 4 or 5, characterized in that the composition includes up to 0.05% by weight of citral.

8. Use according to one of claims 4 to 7, characterized in that the composition includes no formol.

9. Use according to one of claims 1 to 8 in a colored or uncolored nail polish, in a nail polish base coat, or in a base for the care of the nails.

10. Cosmetic composition for the nails including up to 0.05% by weight of citral.
